(19) United States
(12) Patent Application Publication
(10) Pub. No.: US 2021/0015778 A1
(43) Pub. Date: Jan. 21, 2021

(54) Methods and Compositions for the Treatment of Sickle Cell Diseases and Thalassemia

(71) Applicant: Emmaus Medical, Inc., Torrance, CA (US)

(72) Inventor: Yutaka Niihara, Rolling Hills Estates, CA (US)

(73) Assignee: Emmaus Medical, Inc., Torrance, CA (US)

(21) Appl. No.: 16/873,542

(22) Filed: Apr. 30, 2020

Related U.S. Application Data
(62) Division of application No. 16/501,994, filed on Jul. 17, 2019.

(51) Int. Cl. A61K 31/198; A61K 47/36; A61P 7/06

(52) U.S. Cl. CPC A61K 31/198; A61P 7/06; A61K 47/36

(57) ABSTRACT

The present invention is generally directed to methods and compositions for the treatment of sickle cell diseases and thalassemia. It is more particularly directed to compositions containing L-glutamine, an L-glutamine salt or an L-glutamine derivative and at least one other active ingredient and methods using such compositions. In one aspect, the present invention is directed to a composition for treating sickle cell disease or β-thalassemia. The composition comprises: at least one gram of L-glutamine, an L-glutamine salt or an L-glutamine derivative; and, at least one gram of one type of fiber.

This application is a divisional of U.S. patent application Ser. No. 16/501,994, filed Jul. 17, 2019, entitled “Methods and Compositions for the Treatment of Sickle Cell Diseases and Thalassemia”, which is incorporated by reference into this document for all purposes.

### FIELD OF THE INVENTION

The present invention is generally directed to methods and compositions for the treatment of sickle cell diseases and thalassemia. It is more particularly directed to compositions containing L-glutamine, an L-glutamine salt or an L-glutamine derivative and at least one other active ingredient and methods using such compositions.

### BACKGROUND OF THE INVENTION

There have been reports of methods and compositions for the treatment of sickle cell disease. U.S. Pat. No. 10,085,967, entitled “Treatment of sickle cell disease”, for instance, alleges the following: “The present invention includes embodiments for treatment and/or prevention of sickle cell disease that employ Hydroxyfasudil or Isocoronarin D alone or either in conjunction with each other or an inducer of HbF production. The compounds may act synergistically, and the compounds employed circumvent the side effects seen with Hydroxyurea.” Abstract.

U.S. Pat. No. 10,072,067, entitled “Fetal hemoglobin for genetic correction of sickle cell disease”, alleges the following: “Methods and compositions disclosed herein generally relates to methods of determining minimum hematopoietic stem cell (HSC) chimerism and gene dosage for correction of a hematopoietic disease; in particular, in in vivo models. The invention also relates to modified lentiviral expression vectors for increasing a viral titer and various methods for increasing such titers as well as expression vectors capable of enhancing such titers. The invention also relates to CHS4 chromatin insulator-derived functional insulator sequences. The invention also relates to methods for genetic correction of diseases or reducing symptoms thereof, such as sickle cell anemia or β-thalassemia.” Abstract.

U.S. Pat. No. 9,556,266, entitled “Methods of treating sickle cell disease with anti-P-selectin antibodies, alleges the following: The invention features antibodies, e.g., chimeric and humanized antibodies, that recognize (i.e., bind) P-selectin. The P-selectin antibodies prevent P-selectin from binding to its cognate receptor. The P-selectin antibodies can be used to treat inflammatory and thrombotic conditions, e.g., sickle cell disease, pain crisis associated with sickle cell disease, deep vein thrombosis, asthma, rheumatoid arthritis, psoriasis, and ischemia reperfusion injury in a patient in need thereof.” Abstract.

Despite the various reports there is still a need in the art for novel methods and compositions for the treatment of sickle cell diseases and thalassemia.

### SUMMARY OF THE INVENTION

In one aspect, the present invention is directed to a composition for treating sickle cell disease or β-thalassemia. The composition comprises: at least one gram of L-glutamine, an L-glutamine salt or an L-glutamine derivative; and, at least one gram of one type of fiber.

In another aspect, the present invention is directed to a composition for treating sickle cell disease or β-thalassemia. The composition consists essentially of: at least one gram of L-glutamine, an L-glutamine salt or an L-glutamine derivative; and, at least one gram of one type of fiber.

In another aspect, the present invention is directed to a method of treating sickle cell disease or β-thalassemia. The method comprises ingesting a composition, wherein the composition comprises at least one gram of L-glutamine, an L-glutamine salt or an L-glutamine derivative and at least one gram of one type of fiber at a dosage, wherein the dosage ranges from about 0.05 g/kg body weight to about 15.0 g/kg body weight per day, thereby treating sickle cell disease or β-thalassemia.

In another aspect, the present invention is directed to a method of treating sickle cell disease or β-thalassemia. The method comprises ingesting a composition, wherein the composition consists essentially of at least one gram of L-glutamine, an L-glutamine salt or an L-glutamine derivative and at least one gram of one type of fiber at a dosage, wherein the dosage ranges from about 0.05 g/kg body weight to about 15.0 g/kg body weight per day, thereby treating sickle cell disease or β-thalassemia.

### BRIEF DESCRIPTION OF THE DRAWINGS

FIG. 1 shows the structure of L-glutamine.

FIG. 2 shows L-glutamine salts and derivatives.

### DETAILED DESCRIPTION OF THE INVENTION

There are several classifications of fiber, including, without limitation: dietary fiber; functional fiber; total fiber; viscous and nonviscous fiber; fermentable and nonfermentable fiber; and, soluble and insoluble fiber.

Nonlimiting examples of dietary fiber include: lignin; cellulose; β-glucans; hemicelluloses; pectins; gums; inulin and oligofructose; and, resistant starch. Lignin is a polyphenolic compound with a complex three-dimensional structure that is found in the cell walls of woody plants and seeds. Cellulose is a glucose polymer with β-1,4 glycosidic bonds and β-1,3 glycosidic bonds. Hemicelluloses are a diverse group of polysaccharides containing six-carbon sugars and five-carbon sugars. Pectins are viscous polysaccharides that are particularly abundant in fruit and berries. Gums are viscous polysaccharides often found in seeds. Inulin is a mixture of fructose chains that vary in length and often terminate with a glucose molecule. Oligo fructose is a mixture of shorter fructose chains that may terminate in glucose or fructose. Resistant starch is sequestered in plant cell walls and may also be formed by food processing or by cooling and reheating.

Functional fiber consists of isolated nondigestible carbohydrates that have beneficial physiological effects in humans. Nonlimiting examples of functional fiber include: isolated or extracted forms of dietary fibers; psyllium; chitin and chitosan; fructooligosaccharides; polydextrose and polyols; resistant dextrins. Psyllium is viscous mucilage, which is isolated from the husks of psyllium seeds. Chitin is a nondigestible carbohydrate extracted from the exoskeletons of crustaceans. Fructooligosaccharides are short, synthetic fructose chains terminating with a glucose unit. Polydextrose and polyols are synthetic polysaccharides used as bulking agents and sugar substitutes in foods. Resistant dextrins are indigestible polysaccharides formed when starch is heated and treated with enzymes.

Total fiber is the sum of dietary fiber and functional fiber.

Viscous fibers form very viscous solutions or gels in water; non-viscous fibers do not. Viscous fibers include pectins, β-glucans, some gums (e.g., guar gum), and mucilages (e.g., psyllium). Cellulose, lignin and some hemicelluloses are nonviscous fibers.

Fermentable fibers are readily fermented by bacteria that normally colonize the colon; nonfermentable fibers are not. Pectins, β-glucans, guar gum, inulin, and oligofructose are readily fermented, while cellulose and lignin are not.

Soluble fibers are dispersible in water, while insoluble fibers are not. β-Glucans, gums, mucilages (e.g., psyllium), pectins, and some hemicelluloses are soluble fibers, while cellulose, lignin, some pectins, and some hemicelluloses are insoluble fibers.

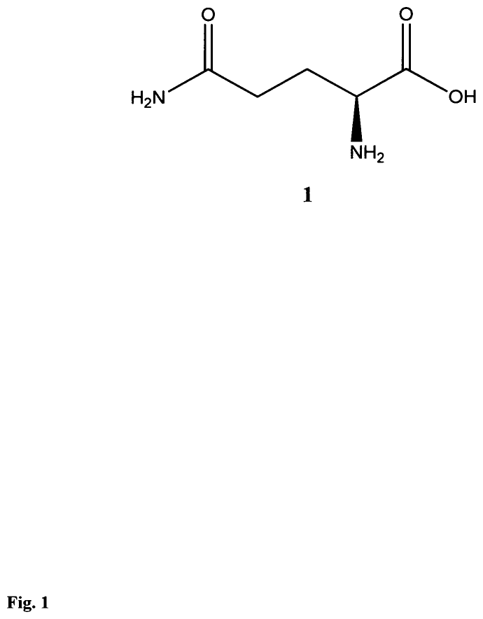

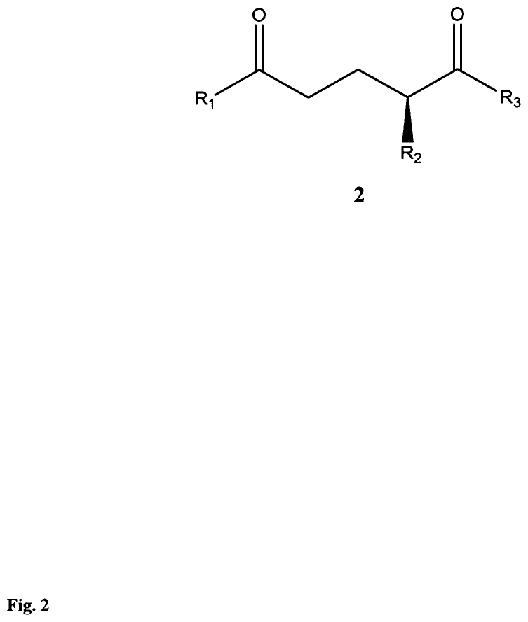

The structure of L-glutamine is shown in FIG. 1, compound 1. L-glutamine salts and derivatives are shown as compound 2 in FIG. 2. Nonlimiting L-glutamine salts and derivatives have the following substituents in reference to compound 2:

R₁ is NH₂;

R₂ is NH₂, NH₃+, NH₃Br, NH₃OPO₃H, NH₃OC(O)CH₃ (i.e., acetate salt), NH₃OC(O)CHCHCO₂H (i.e., fumarate salt—trans olefin), NH₃OC(O)CH(OH)CH(OH)CO₂H (i.e., tartrate salt), NH₃OC(O)CHCHCO₂H (i.e., maleate salt—cis olefin), NH₃OC(O)CH₂—C(OH)(CO₂H)CH₂CO₂H (i.e., citrate salt), NH₃OC(O)CO₂H (i.e., oxalate salt), NH₃OS(O)₂OH (i.e., methansulfonate salt), NH₃OS(O)₂C₆H₄CH₃ (i.e., p-toluenesulfonate salt), and, NH₃OC(O)C(O)CH₂CH₂CO₂H (i.e., alpha-ketoglutarate salt).

R₃ is OH, O⁻, ONa, OK, OCa, OLi, ONH₂(CH₂C₆H₅)CH₂CH₂NHCH₂C₆H₅ (i.e., benzathine salt), ON(CH₂CH₃)₂CH₂CH₂OC(O)C₆H₃ClNH₂ (i.e., chloroprocaine salt), ON(CH₃)₃CH₂CH₂OH (i.e., choline salt), ONH₂(CH₂CH₂OH)₂ (i.e., diethanolamine salt), ONH₃CH₂CH₂OH (i.e., ethanolamine salt), ONH₃CH₂CH₂NH₂ (i.e., ethyldiamine salt), ONH₂(CH₃)CH₂CH(OH)CH(OH)CH(OH)CH(OH)CH₂OH (i.e., meglumine salt), ONH₃C(CH₂OH)₃ (i.e., tromethamine salt), ONH₃C(CH₃)₃ (i.e., tertiary-butylamine salt), ON(CH₂CH₃)₂CH₂CH₂OC(O)C₆H₄NH₂ (i.e., procaine salt), NHCH(CH₃)CO₂H, NHCH(CH(CH₃)₂)CO₂H, NHCH(CH(CH₃)(CH₂CH₃))CO₂H, NHCH(CH₂CH(CH₃)₂)CO₂H, NHCH(CH₂CH₂SCH₃)CO₂H, NHCH(CH₂C₆H₅)CO₂H, NHCH(CH₂C₆H₅OH)CO₂H, NHCH(CH₂C₈H₆N)CO₂H, NHCH₂CO₂H, NHCH(CH₂CH₂C(O)NH₂)CO₂H, NHCH(CH₂C(O)NH₂)CO₂H, NHCH(CH(OH)CH₃)CO₂H, NHCH(CH₂OH)CO₂H, NHCH(CH₂CH₂CO₂H)CO₂H, NHCH(CH₂CO₂H)CO₂H, NHC(O)CH(NH₂)CH₃.

The transitional term “comprising”, which is synonymous with “including,” “containing,” or “characterized by,” is inclusive or open-ended and does not exclude additional, unrecited elements or method steps.

The transitional phrase “consisting essentially of” limits the scope of a claim to the specified materials or steps “and those that do not materially affect the basic and novel characteristic(s)” of the claimed invention.

The transitional phrase “consisting of” excludes any element, step, or ingredient not specified in the claim.

Compositions according to the present invention are typically one of the following types: a composition comprising L-glutamine, an L-glutamine salt or an L-glutamine derivative and at least one type of fiber (the composition may include one or more additional active ingredients); a composition consisting essentially of L-glutamine, an L-glutamine salt or an L-glutamine derivate and at least one type of fiber (the composition includes additional ingredients that do not materially affect the treatment of sickle cell disease, e.g., an excipient); a composition consisting of L-glutamine, an L-glutamine salt or an L-glutamine derivative and at least one type of fiber (the composition only includes the listed ingredients).

The compositions typically include at least one gram of L-glutamine, an L-glutamine salt or an L-glutamine derivative and at least one gram of fiber. In certain cases, the compositions include at least about 2.5 g of L-glutamine, an L-glutamine salt or an L-glutamine derivative and at least about 2.5 g of fiber, at least about 5.0 g of L-glutamine, an L-glutamine salt or an L-glutamine derivative and at least about 5.0 g of fiber, or at least about 10.0 g of L-glutamine, an L-glutamine salt or an L-glutamine derivative and at least about 10.0 g of fiber.

The following are examples of compositions according to the present invention: 1) A composition comprising L-glutamine and one type of fiber. 2) A composition comprising L-glutamine and one type of dietary fiber. 3) A composition comprising L-glutamine and one type of functional fiber. 4) A composition comprising L-glutamine and one type of viscous fiber. 5) A composition comprising L-glutamine and one type of nonviscous fiber. 6) A composition comprising L-glutamine and one type of fermentable fiber. 7) A composition comprising L-glutamine and one type of nonfermentable fiber. 8) A composition comprising L-glutamine and at least one type of soluble fiber. 9) A composition comprising L-glutamine and at least one type of insoluble fiber.

The following are further examples of compositions according to the present invention: 10) A composition comprising L-glutamine and more than one type of fiber. 11) A composition comprising L-glutamine and more than one type of fiber, where one type of fiber is dietary fiber. 12) A composition comprising L-glutamine and more than one type of fiber, where one type of fiber is functional fiber. 13) A composition comprising L-glutamine and more than one type of fiber, where one type of fiber is viscous fiber. 14) A composition comprising L-glutamine and more than one type of fiber, where one type of fiber is nonviscous fiber. 15) A composition comprising L-glutamine and more than one type of fiber, where one type of fiber is fermentable fiber. 16) A composition comprising L-glutamine and more than one type of fiber, where one type of fiber is nonfermentable fiber. 17) A composition comprising L-glutamine and more than one type of fiber, where one type of fiber is soluble fiber. 18) A composition comprising L-glutamine and more than one type of fiber, where one type of fiber is insoluble fiber.

The following are further examples of compositions according to the present invention: 19) A composition comprising L-glutamine and a soluble fiber, where the soluble fiber is a β-Glucan. 20) A composition comprising L-glutamine and a soluble fiber, where the soluble fiber is a gum. 21) A composition comprising L-glutamine and a soluble fiber, where the soluble fiber is a mucilage, preferably psyllium. 22) A composition comprising L-glutamine and a soluble fiber, where the soluble fiber is a pectin. 23) A composition comprising L-glutamine and a soluble fiber, where the soluble fiber is a hemicellulose.

The following are further examples of compositions according to the present invention: 24) A composition consisting essentially of L-glutamine and one type of fiber, where an included non-material ingredient is an excipient (e.g., water, juice, a flavorant, a colorant, a buffer). 25) A composition consisting essentially of L-glutamine and one type of dietary fiber, where an included non-material ingredient is an excipient (e.g., water, juice, a flavorant, a colorant, a buffer). 26) A composition consisting essentially of L-glutamine and one type of functional fiber, where an included non-material ingredient is an excipient (e.g., water, juice, a flavorant, a colorant, a buffer). 27) A composition consisting essentially of L-glutamine and one type of viscous fiber, where an included non-material ingredient is an excipient (e.g., water, juice, a flavorant, a colorant, a buffer). 28) A composition consisting essentially of L-glutamine and one type of nonviscous fiber, where an included non-material ingredient is an excipient (e.g., water, juice, a flavorant, a colorant, a buffer). 29) A composition consisting essentially of L-glutamine and one type of fermentable fiber, where an included non-material ingredient is an excipient (e.g., water, juice, a flavorant, a colorant, a buffer). 30) A composition consisting essentially of L-glutamine and one type of nonfermentable fiber, where an included non-material ingredient is an excipient (e.g., water, juice, a flavorant, a colorant, a buffer). 31) A composition consisting essentially of L-glutamine and at least one type of soluble fiber, where an included non-material ingredient is an excipient (e.g., water, juice, a flavorant, a colorant, a buffer). 32) A composition consisting essentially of L-glutamine and at least one type of insoluble fiber, where an included non-material ingredient is an excipient (e.g., water, juice, a flavorant, a colorant, a buffer).

The following are further examples of compositions according to the present invention: 33) A composition consisting essentially of L-glutamine and more than one type of fiber, where an included non-material ingredient is an excipient (e.g., water, juice, a flavorant, a colorant, a buffer). 34) A composition consisting essentially of L-glutamine and more than one type of fiber, where one type of fiber is dietary fiber, and where an included non-material ingredient is an excipient (e.g., water, juice, a flavorant, a colorant, a buffer). 35) A composition consisting essentially of L-glutamine and more than one type of fiber, where one type of fiber is functional fiber, and where an included non-material ingredient is an excipient (e.g., water, juice, a flavorant, a colorant, a buffer). 36) A composition consisting essentially of L-glutamine and more than one type of fiber, where one type of fiber is viscous fiber, and where an included non-material ingredient is an excipient (e.g., water, juice, a flavorant, a colorant, a buffer). 37) A composition consisting essentially of L-glutamine and more than one type of fiber, where one type of fiber is nonviscous fiber, and where an included non-material ingredient is an excipient (e.g., water, juice, a flavorant, a colorant, a buffer). 38) A composition consisting essentially of L-glutamine and more than one type of fiber, where one type of fiber is fermentable fiber, and where an included non-material ingredient is an excipient (e.g., water, juice, a flavorant, a colorant, a buffer). 39) A composition consisting essentially of L-glutamine and more than one type of fiber, where one type of fiber is nonfermentable fiber, and where an included non-material ingredient is an excipient (e.g., water, juice, a flavorant, a colorant, a buffer). 40) A composition consisting essentially of L-glutamine and more than one type of fiber, where one type of fiber is soluble fiber, and where an included non-material ingredient is an excipient (e.g., water, juice, a flavorant, a colorant, a buffer). 41) A composition consisting essentially of L-glutamine and more than one type of fiber, where one type of fiber is insoluble fiber, and where an included non-material ingredient is an excipient (e.g., water, juice, a flavorant, a colorant, a buffer).

The following are further examples of compositions according to the present invention: 42) A composition consisting essentially of L-glutamine and a soluble fiber, where the soluble fiber is a β-Glucan, and where an included non-material ingredient is an excipient (e.g., water, juice, a flavorant, a colorant, a buffer). 43) A composition consisting essentially of L-glutamine and a soluble fiber, where the soluble fiber is a gum, and where an included non-material ingredient is an excipient (e.g., water, juice, a flavorant, a colorant, a buffer). 44) A composition consisting essentially of L-glutamine and a soluble fiber, where the soluble fiber is a mucilage, preferably psyllium, and where an included non-material ingredient is an excipient (e.g., water, juice, a flavorant, a colorant, a buffer). 45) A composition consisting essentially of L-glutamine and a soluble fiber, where the soluble fiber is a pectin, and where an included non-material ingredient is an excipient (e.g., water, juice, a flavorant, a colorant, a buffer). 46) A composition consisting essentially of L-glutamine and a soluble fiber, where the soluble fiber is a hemicellulose, and where an included non-material ingredient is an excipient (e.g., water, juice, a flavorant, a colorant, a buffer).

The following are examples of compositions according to the present invention: 47) A composition consisting of L-glutamine and one type of fiber. 48) A composition consisting of L-glutamine and one type of dietary fiber. 49) A composition consisting of L-glutamine and one type of functional fiber. 50) A composition consisting of L-glutamine and one type of viscous fiber. 51) A composition consisting of L-glutamine and one type of nonviscous fiber. 52) A composition consisting of L-glutamine and one type of fermentable fiber. 53) A composition consisting of L-glutamine and one type of nonfermentable fiber. 54) A composition consisting of L-glutamine and at least one type of soluble fiber. 55) A composition consisting of L-glutamine and at least one type of insoluble fiber.

The following are further examples of compositions according to the present invention: 56) A composition consisting of L-glutamine and more than one type of fiber. 57) A composition consisting of L-glutamine and more than one type of fiber, where one type of fiber is dietary fiber. 58) A composition consisting of L-glutamine and more than one type of fiber, where one type of fiber is functional fiber. 59) A composition consisting of L-glutamine and more than one type of fiber, where one type of fiber is viscous fiber. 60) A composition consisting of L-glutamine and more than one type of fiber, where one type of fiber is nonviscous fiber. 61) A composition consisting of L-glutamine and more than one type of fiber, where one type of fiber is fermentable fiber. 62) A composition consisting of L-glutamine and more than one type of fiber, where one type of fiber is nonfermentable fiber. 63) A composition consisting of L-glutamine and more than one type of fiber, where one type of fiber is soluble fiber. 64) A composition consisting of L-glutamine and more than one type of fiber, where one type of fiber is insoluble fiber.

The following are further examples of compositions according to the present invention: 65) A composition consisting of L-glutamine and a soluble fiber, where the soluble fiber is a 13-Glucan. 66) A composition consisting of L-glutamine and a soluble fiber, where the soluble fiber is a gum. 67) A composition consisting of L-glutamine and a soluble fiber, where the soluble fiber is a mucilage, preferably psyllium. 68) A composition consisting of L-glutamine and a soluble fiber, where the soluble fiber is a pectin. 69) A composition consisting of L-glutamine and a soluble fiber, where the soluble fiber is a hemicellulose.

The following are examples of compositions according to the present invention: 70) A composition comprising an L-glutamine salt and one type of fiber. 71) A composition comprising an L-glutamine salt and one type of dietary fiber. 72) A composition comprising an L-glutamine salt and one type of functional fiber. 73) A composition comprising an L-glutamine salt and one type of viscous fiber. 74) A composition comprising an L-glutamine salt and one type of nonviscous fiber. 75) A composition comprising an L-glutamine salt and one type of fermentable fiber. 76) A composition comprising an L-glutamine salt and one type of nonfermentable fiber. 77) A composition comprising an L-glutamine salt and at least one type of soluble fiber. 78) A composition comprising an L-glutamine salt and at least one type of insoluble fiber.

The following are further examples of compositions according to the present invention: 79) A composition comprising an L-glutamine salt and more than one type of fiber. 80) A composition comprising an L-glutamine salt and more than one type of fiber, where one type of fiber is dietary fiber. 81) A composition comprising an L-glutamine salt and more than one type of fiber, where one type of fiber is functional fiber. 82) A composition comprising an L-glutamine salt and more than one type of fiber, where one type of fiber is viscous fiber. 83) A composition comprising an L-glutamine salt and more than one type of fiber, where one type of fiber is nonviscous fiber. 84) A composition comprising an L-glutamine salt and more than one type of fiber, where one type of fiber is fermentable fiber. 85) A composition comprising an L-glutamine salt and more than one type of fiber, where one type of fiber is nonfermentable fiber. 86) A composition comprising an L-glutamine salt and more than one type of fiber, where one type of fiber is soluble fiber. 87) A composition comprising an L-glutamine salt and more than one type of fiber, where one type of fiber is insoluble fiber.

The following are further examples of compositions according to the present invention: 88. A composition comprising an L-glutamine salt and a soluble fiber, where the soluble fiber is a β-Glucan. 89. A composition comprising an L-glutamine salt and a soluble fiber, where the soluble fiber is a gum. 90. A composition comprising an L-glutamine salt and a soluble fiber, where the soluble fiber is a mucilage, preferably psyllium. 91. A composition comprising an L-glutamine salt and a soluble fiber, where the soluble fiber is a pectin. 92. A composition comprising an L-glutamine salt and a soluble fiber, where the soluble fiber is a hemicellulose.

Excipients are inactive substances that serves as a vehicle or medium for a drug or other active substance. Nonlimiting examples of excipients include: water; juices (e.g., orange juice); buffers; flavorants; and colorants.

Further nonlimiting examples of excipients include: acacia; acesulfame potassium; acetic acid, glacial; acetone; acetyltributyl citrate; acetyltriethyl citrate; adipic acid; albumin; alcohol; alginic acid; aliphatic polyesters; alitame; almond oil; alpha tocopherol; aluminum hydroxide adjuvant; aluminum monostearate; aluminum oxide; aluminum phosphate adjuvant; ammonia solution; ammonium chloride; ascorbic acid; ascorbyl palmitate; aspartame; attapulgite; bentonite; benzakonium chloride; benzethonium chloride; benzoic acid; benzyl alcohol; benzyl benzoate; boric acid; bronopol; butylated hydroxyanisole; butylated hydroxytoluene; butylene glycol; butylparaben; calcium acetate; calcium carbonate; calcium chloride; calcium hydroxide; calcium lactate; calcium phosphate, dibasic anhydrous; calcium phosphate, dibasic dihydrate; calcium phosphate, tribasic; calcium silicate; calcium stearate; calcium sulfate; canola oil; carbomer; carbon dioxide; castor oil; castor oil, hydrogenated; ceratonia; ceresin; cetostearyl alcohol; cetrimide; cetyl alcohol; cetylpyridinium chloride; chlorhexidine; chlorobutanol; chlorocresol; chlorodifluoroethane (HCFC); chlorofluorocarbons (CFC); chloroxylenol; cholesterol; citric acid monohydrate; coconut oil; colloidal silicon dioxide; and, coloring agents.

Further nonlimiting examples of excipients include: capovidone; corn oil; cottonseed oil; cresol; croscarmellose sodium; crospovidone; cyclomethicone; denatonium benzoate; dextrates; dextrose; dibutyl phthalate; dibutyl sebacate; diethanolamine; diethyl phthalate; difluoroethane (HFC); dimethicone; dimethyl ether; dimethyl phthalate; dimethyl sulfoxide; dimethylacetamide; disodium edetate; docusate sodium; edetic acid; erythorbic acid; erythritol; ethyl acetate; ethyl lactate; ethyl maltol; ethyl oleate; ethyl vanillin; ethylene glycol stearates; ethylene vinyl acetate; ethylparaben; fumaric acid; gelatin; glucose, liquid; glycerin; glyceryl behenate; glyceryl monooleate; glyceryl monostearate; glyceryl palmitostearate; glycine; glycofural; hectorite; heptafluoropropane (HFC); hexetidine; hydrocarbons (HC); hydrochloric acid; hydrophobic colloidal silica; hypromellose; hypromellose acetate succinate; hypromellose phthalate; imidurea; iron oxides; isomalt; isopropyl alcohol; isopropyl myristate; isopropyl palmitate; kaolin; lactic acid; lactitol; lactose, anhydrous; lactose, inhalation; lactose monohydrate; lactose, monohydrate and povidone; lactose, spray-dried; lanolin; lanolin hydrous; and, lanolin alcohols.

Further nonlimiting examples of excipients include: lauric acid; lecithin; leucine; linoleic acid; macrogol 15 hydroxystearate; magnesium aluminum silicate; magnesium carbonate; magnesium oxide; magnesium silicate; magnesium stearate; magnesium trisilicate; maleic acid; malic acid; maltitol; maltitol solution; maltol; maltose; mannitol; medium-chain triglycerides; meglumine; menthol; methionine; methylparaben; mineral oil; mineral oil, light; mineral oil and lanolin alcohols; monoethanolamine; monosodium glutamate; monothioglycerol; myristic acid; myristyl alcohol; neohesperidin dihydrochalcone; neotame; nitrogen; nitrous oxide; octyldodecanol; oleic acid; oleyl alcohol; olive oil; palmitic acid; paraffin; peanut oil; pentetic acid; petrolatum; petrolatum and lanolin alcohols; phenol; phenoxyethanol; phenylethyl alcohol; phenylmercuric acetate; phenylmercuric borate; phenylmercuric nitrate; phospholipids; phosphoric acid; polacrilin potassium; poloxamer; polycarbophil; poly (DL-lactic acid); polyethylene glycol; polyethylene oxide; polymethacrylates; poly(methyl vinyl ether/maleic anhydride); polyoxyethylene alkyl ethers; polyoxyethylene castor oil derivates; polyoxyethylene sorbitan fatty acid esters; polyoxyethylene stearates; and, polyoxylglycerides.

Further nonlimiting examples of excipients include: polyvinyl acetate phthalate; polyvinyl alcohol; potassium alum; potassium benzoate; potassium bicarbonate; potassium chloride; potassium citrate; potassium hydroxide; potassium metabisulfite; potassium sorbate; povidone; propionic acid; propyl gallate; propylene carbonate; propylene glycol; propylparaben;

propylparaben sodium; pyrrolidone; saccharin; saccharin sodium; safflower oil; saponite; sesame oil; shellac; simethicone; sodium acetate; sodium ascorbate; sodium benzoate; sodium bicarbonate; sodium borate; sodium carbonate; sodium chloride; sodium citrate dihydrate; sodium cyclamate; sodium formaldehyde sulfoxylate; sodium hyaluronate; sodium hydroxide; sodium lactate; sodium lauryl sulfate; sodium metabisulfite; sodium phosphate, dibasic; sodium phosphate, monobasic; sodium propionate; sodium stearyl fumarate; sodium sulfite; sodium thiosulfate; sorbic acid; sorbitan esters (sorbitan fatty acid esters); and, sorbitol. Further nonlimiting examples of excipients include: soybean oil; stearic acid; stearyl alcohol; sucralose; sucrose; sucrose octaacetate; sugar, compressible; sugar, confectioner's; sugar spheres; sulfur dioxide; sulfuric acid; sunflower oil; suppository bases, hard fat; tagatose; talc; tartaric acid; tetrafluoroethane (HFC); thaumatin; thimerosal; thymol; titanium dioxide; tragacanth; tehalose; triacetin; tributyl citrate; tricaprylin; triethanolamine; triethyl citrate; triolein; vanillin; vegetable oil, hydrogenated; vitamin E polyethylene glycol succinate; water; wax, anionic emulsifying; wax, carnauba; wax, cetyl esers; wax, microcrystalline; wax, nonionic emulsifying; wax, white; wax, yellow; xylitol; zein; zinc acetate; and, zinc stearate

The present invention provides methods of treating sickle cell diseases and thalassemia. The method involves ingestion/oral administration of a composition of the present invention, where the composition is one of the compositions “1” through “92”, including “1” and “92”, as listed above. The amount of L-glutamine, salts or derivatives in the composition typically ranges from about 0.05 g/kg body weight to about 15.0 g/kg body weight. Oftentimes, the amount ranges from about 0.075 g/kg body weight to about 12.5 g/kg body weight, from about 0.10 g/kg body weight to about 10.0 g/kg body weight, from about 0.125 g/kg body weight to about 7.5 g/kg body weight, or from about 0.15 g/kg body weight to about 5.0 g/kg body weight. The amount of fiber in the composition typically ranges from about 0.05 g/kg body weight to about 15.0 g/kg body weight. Oftentimes, the amount ranges from about 0.075 g/kg body weight to about 12.5 g/kg body weight, from about 0.10 g/kg body weight to about 10.0 g/kg body weight, from about 0.125 g/kg body weight to about 7.5 g/kg body weight, or from about 0.15 g/kg body weight to about 5.0 g/kg body weight.

Ingestion/oral administration of the composition of the present invention typically occurs once per day. The composition may, however, be administered more than once per day where indicated. The amount of composition ingested/orally administered during a day may also be divided and taken at times throughout the day (e.g., ⅓ of daily dose taken at morning, afternoon and evening).

The composition is typically ingested by/administered to a patient having sickle cell disease or β-thalassemia for a period longer than about one week. In certain cases, the composition is ingested by/administered to a patient for a period longer than about 1 month, for a period longer than about 3 months, for a period longer than about 6 months, for a period longer than about 9 months, for a period longer than about 12 months, for a period longer than about 18 months, for a period longer than about 24 months, for a period longer than about 36 months or for a period longer than about 60 months.

The L-glutamine, L-glutamine salt or L-glutamine derivative is typically ingested/administered in water, although any suitable delivery medium is acceptable (e.g., juice, smoothie, bar, other food etc.). The medium is typically unheated.

Results or efficacy of ingesting/orally administering compositions of the present invention by/to patients having sickle cell anemia or sickle β-thalassemia can be assessed using methods reported in the New England Journal of Medicine, as follows: A year-long randomized, placebo-controlled, double-blind, parallel-group trial may be conducted. Patients would be eligible for enrollment if they are at least 5 years of age, have received a diagnosis of sickle cell anemia or sickle β-thalassemia and have had at least two pain crises (no upper limit) documented during the previous year. A pain crisis would be defined as pain leading to treatment with a parenterally administered narcotic or ketorolac in an emergency department (or outpatient treatment center) or during hospitalization. Patients receiving treatment with hydroxyurea, or L-glutamine by itself, at a dose that had been stable for at least 3 months before screening and who intended to continue that treatment would be eligible to participate. Women of childbearing potential would be permitted if they agreed to use contraception during the study.

Patients would be excluded if they had been hospitalized for a reason not related to sickle cell disease within 2 months before screening, have a prothrombin-time international normalized ratio higher than 2.0, have a serum albumin level of less than 3.0 g per deciliter, have received any blood products within 3 weeks before screening, or have clinically significant renal or liver disease. Eligible patients would be randomly assigned, in a 2:1 ratio, to receive a composition according to the present invention or placebo, with randomization stratified according to region of participating site and status with respect to hydroxyurea use. The planned treatment period would be 48 weeks, during which patients would receive a composition according to the present invention or placebo powder (e.g., 100% maltodextrin) such that it would be ingested/administered twice daily.

The primary efficacy end point would be the number of pain crises through week 48. Acute chest syndrome, priapism, and splenic sequestration would be classified as sickle cell-related events regardless of the need for narcotics or ketorolac. Secondary efficacy end points would include the number of hospitalizations for sickle cell-related pain, the number of visits to an ED (or outpatient treatment center) for sickle cell-related pain, and changes in hematologic measures (hemoglobin and hematocrit levels and reticulocyte count) from baseline through week 48. A visit to an ED (or outpatient treatment center) on the same calendar day as a hospital admission would be counted only as a hospitalization.

All reported pain crises would be recorded on case-report forms. An independent adjudication committee consisting of hematology-oncology physicians who were unaware of the composition/placebo assignments would evaluate each episode to determine whether the event met the definition of a pain crisis for the efficacy evaluation.

Coagulation screening, hemoglobin electrophoresis, human immunodeficiency virus tests, and pregnancy tests would be performed, and the findings would be evaluated at screening. Blood samples would be obtained at screening and at the end of the study for serum chemical testing and at monthly trial visits for complete blood counts with reticulocyte counts; no dose adjustments based on results of laboratory tests would be required by the protocol. See, Niihara et al. N Engl. J Med. 2018; 379: 226-235.

Sickle cell disease or β-thalassemia patients ingesting/receiving a composition according to the present invention should typically experience fewer pain crises and fewer hospitalizations than patients receiving L-glutamine, L-glutamine salts or L-glutamine derivatives alone (i.e., without fiber). For instance, oftentimes at least about 5 percent fewer pain crises, at least about 10 percent fewer pain crises, at least about 15 percent fewer pain crises, at least about 20 percent fewer pain crises, or at least about 25 percent fewer pain crises should be reported; at least about 5 percent fewer hospitalizations, at least about 10 percent fewer hospitalizations, at least about 15 percent fewer hospitalizations, at least about 20 percent fewer hospitalizations, or at least about 25 percent fewer hospitalizations should be reported.

1. A composition for treating sickle cell disease or β-thalassemia, wherein the composition comprises:
a) at least one gram of L-glutamine, an L-glutamine salt or an L-glutamine derivative;
b) at least one gram of one type of fiber.

2. The composition according to claim 1, wherein the composition comprises L-glutamine.

3. The composition according to claim 1, wherein the composition comprises a soluble fiber.

4. The composition according to claim 2, wherein the composition comprises a soluble fiber.

5. The composition according to claim 4, wherein the soluble fiber is selected from a group of soluble fibers consisting of guar gum and psyllium.

6. A composition for treating sickle cell disease or β-thalassemia, wherein the composition consists essentially of:
a) at least one gram of L-glutamine, an L-glutamine salt or an L-glutamine derivative;
b) at least one gram of one type of fiber.

7. The composition according to claim 6, wherein the composition consists essentially of at least one gram of L-glutamine and at least one gram of one type of fiber.

8. The composition according to claim 6, wherein the composition consists essentially of at least one gram of L-glutamine, an L-glutamine salt or an L-glutamine derivative and at least one gram of a soluble fiber.

9. The composition according to claim 7, wherein the at least one gram of fiber is soluble fiber.

10. The composition according to claim 9, wherein the soluble fiber is selected from a group of soluble fibers consisting of guar gum and psyllium.
